(19) United States
(12) Patent Application Publication
(10) Pub. No.: US 2023/0287181 A1
(43) Pub. Date: Sep. 14, 2023

(54) ZWITTERIONIC COMPOUND, PRODUCING METHOD THEREOF, POLYMER THEREOF, AND CONDUCTIVE STRUCTURE

(71) Applicant: National Yang Ming Chiao Tung University, Hsinchu City (TW)

(72) Inventors: Hsin-Chieh Lin, Hsinchu City (TW); Satish Kumar Talloj, Hsinchu City (TW)

(73) Assignee: National Yang Ming Chiao Tung University, Hsinchu City (TW)

(21) Appl. No.: 18/119,420

(22) Filed: May 26, 2023

Related U.S. Application Data
(60) Provisional application No. 63/269,111, filed on Mar. 10, 2022.

(51) Int. Cl. C08G 75/06; C07D 333/32; C07D 409/14; C07F 9/6553; C07F 9/6558

(52) U.S. Cl. CPC C08G 75/06; C07D 333/32; C07D 409/14; C07F 9/655345; C07F 9/65586

(57) ABSTRACT

A zwitterionic compound has a structure of following formula (1), formula (2), formula (3), or formula (4):

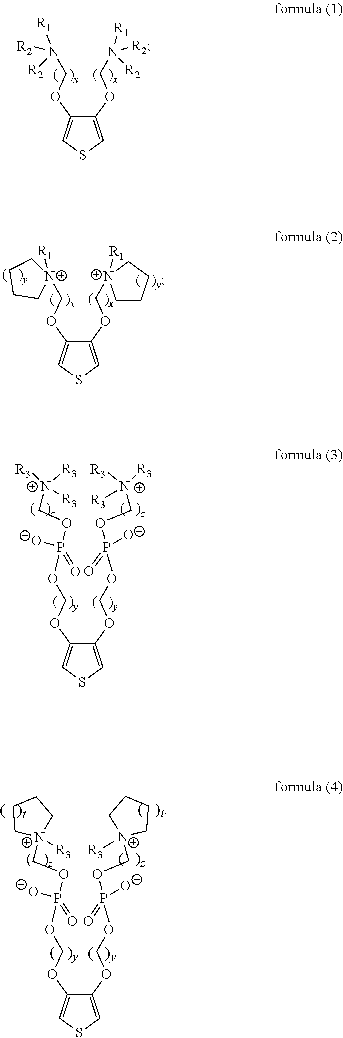

R₁ is —O⁻, —(CH₂)ₐSO₃⁻, or —(CH₂)_{b}CO₂⁻, a is 1 to 10, and b is 1 to 10. R₂ and R₃ are each independently an alkyl group or a phenyl group. x, z, and v are each independently 1 to 20. y and t are each independently 0 to 10.

### CROSS-REFERENCE TO RELATED APPLICATION

This application claims priority to U.S. Provisional Application Ser. No. 63/269,111, filed Mar. 10, 2022, which is herein incorporated by reference.

### BACKGROUND

### Field of Invention

The present disclosure relates to a zwitterionic compound, a zwitterionic polymer, a conductive structure, and a method of producing a zwitterionic compound.

### Description of Related Art

In recent years, implantable medical devices have been booming, and their global market has been growing rapidly. In clinical applications, the implantable medical devices can be used to diagnose a variety of diseases, such as detecting cancer and monitoring chronic diseases. The implantable medical devices often use electrodes and probes made of conductive materials to convert biological signals in living organisms into interpretable signals. However, the electrodes and probes are prone to biofouling when they come into contact with tissues in the organism, which may cause the implantable devices to malfunction or fail. Therefore, in order to extend the service life of the electrodes, probes, and implantable devices, it is necessary to enhance the resistance of the electrode surfaces and probe surfaces to nonspecific biofouling. The formation of modified coatings on the electrode surfaces and probe surfaces has been developed to enhance the anti-biofouling ability. However, there are still some problems with the existing modified coatings materials, such as high price or poor anti-biofouling effect.

In view of the above, it is necessary to provide a new modified coating material to reduce the manufacturing cost of the modified coating and enhance its anti-biofouling ability.

### SUMMARY

Some embodiments of the present disclosure provide a zwitterionic compound having a structure of following formula (1), formula (2), formula (3), or formula (4):

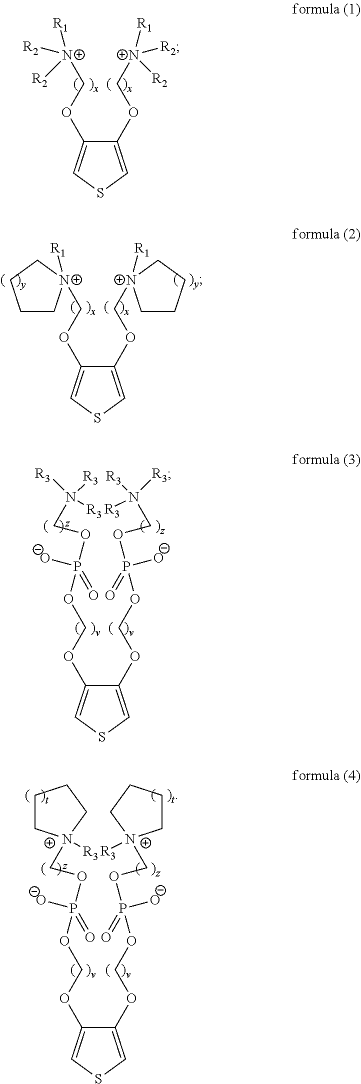

R₁ is —O⁻, —(CH₂)ₐSO₃⁻, or —(CH₂)_{b}CO₂⁻, a is 1 to 10, and b is 1 to 10. R₂ and R₃ are each independently an alkyl group or a phenyl group. x, z, and v are each independently 1 to 20. y and t are each independently 0 to 10.

In some embodiments, the zwitterionic compound has the structure of formula (1), R₁ is —O⁻, R₂ is a methyl group, and x is 2.

In some embodiments, the zwitterionic compound has the structure of formula (1), R₁ is —(CH₂)ₐSO₃⁻, a is 3, R₂ is a methyl group, and x is 2.

Some embodiments of the present disclosure provide a zwitterionic polymer including a chain segment formed by the zwitterionic compound having the formula (1), the zwitterionic compound having the formula (2), the zwitterionic compound having the formula (3), the zwitterionic compound having the formula (4), of any of the above embodiments, or combinations thereof. The zwitterionic polymer has a structure of following formula (5), formula (6), formula (7), formula (8), or combinations thereof:

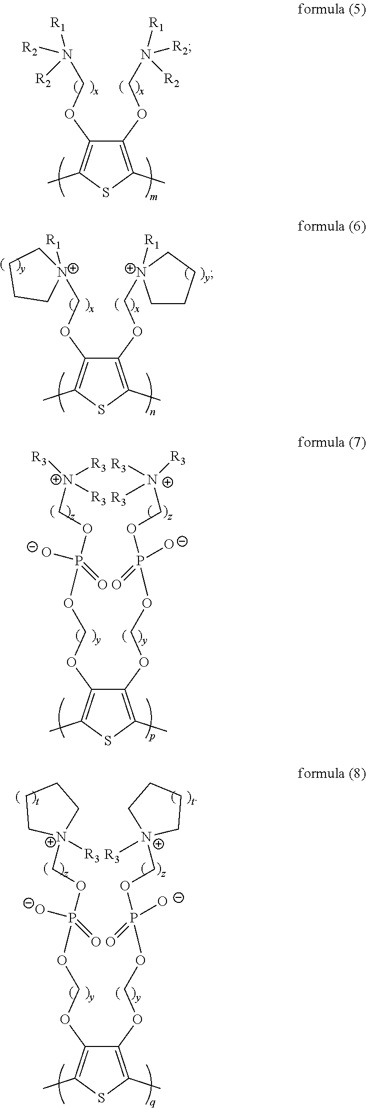

in which m is 1 to 10000, n is 1 to 10000, p is 1 to 10000, and q is 1 to 10000.

Some embodiments of the present disclosure provide a conductive structure including a conductive substrate and a conductive film. The conductive substrate includes a metal, an alloy, a metal oxide, a semiconductor material, a carbon material, or combinations thereof. The conductive film covers the conductive substrate, in which the conductive film includes the zwitterionic polymer of the above embodiments.

Some embodiments of the present disclosure provide a method of producing a zwitterionic compound. The method includes reacting an oxidizing agent, a lactone, or a sultone with a reactant to obtain the zwitterionic compound having the structure of formula (1) or formula (2), in which the reactant has a structure of following formula (9) or formula (10):

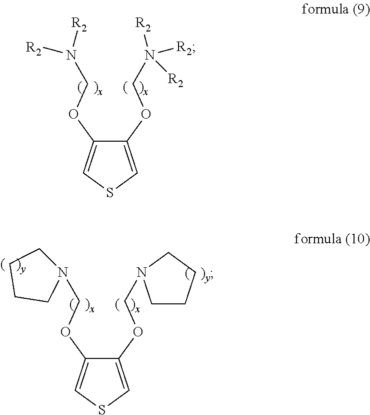

In some embodiments, the oxidizing agent includes meta-chloroperoxybenzoic acid (mCPBA), hydrogen peroxide, or a combination thereof.

In some embodiments, reacting the oxidizing agent with the reactant is performed at a temperature of 20° C. to 35° C.

In some embodiments, when reacting the lactone or the sultone with the reactant, the lactone has a carbon number of 2 to 11, and the sultone has a carbon number of 1 to 10.

In some embodiments, reacting the lactone or the sultone with the reactant is performed at a temperature of 40° C. to 60° C.

In some embodiments, R₂ is a methyl group, and x is 2.

Some embodiments of the present disclosure provide a method of producing a zwitterionic compound, and the method includes the following operations. 3,4-dibromothiophene is reacted with

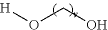

to form a first compound. The first compound is reacted with

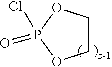

to form a second compound. The second compound is reacted with an amine to obtain the zwitterionic compound having the structure of formula (3) or formula (4), in which the amine has a structure of

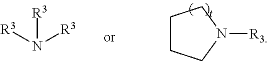

In some embodiments, reacting the 3,4-dibromothiophene with

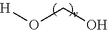

includes dissolving the 3,4-dibromothiophene,

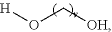

and a catalyst in a solvent, in which the catalyst is copper(I) iodide.

It is to be understood that the foregoing general description and the following detailed description are merely exemplary and explanatory, and are intended to provide further illustration of the present disclosure.

### BRIEF DESCRIPTION OF THE DRAWINGS

The above contents of the present disclosure, other aspects, features, and other advantages are referred to the contents of the specification and with attached drawings for a clearer understanding.

FIG. 1 is a cross-sectional view of a conductive structure in accordance with various embodiments of the present disclosure.

FIG. 2 shows the test results of the anti-cell adhesion ability in accordance with the examples and comparative examples of the present disclosure.

### DETAILED DESCRIPTION

In order to make the description of the present disclosure more detailed and complete, the following provides an illustrative description of the embodiments and specific examples of the invention: however, this is not the only form in which specific embodiments of the invention are implemented or applied. The embodiments disclosed below may be combined or substituted for each other under useful circumstances, and other embodiments may be added to some embodiments without further recitation or description.

The present disclosure relates to a zwitterionic compound and a method for producing the same. Moreover, zwitterionic compounds can be used to prepare a zwitterionic polymer. In other words, the zwitterionic compounds can be used as monomers for synthesizing a zwitterionic polymer. The zwitterionic compound of the present disclosure has two hydrophilic zwitterionic groups, which can make the zwitterionic compound highly hydrophilic and simultaneously improve its anti-biofouling ability or anti-fouling ability. The zwitterionic compound contains a thiophene ring, so the zwitterionic polymer has good conductivity. Moreover, since reactants for synthesizing the zwitterionic compound are cheap, the zwitterionic compound and polymer of the present disclosure have the advantage of low cost. The zwitterionic polymer can have high hydrophilicity, high electrical conductivity, high anti-biofouling ability, and high biocompatibility, and have a low immunogenicity. The zwitterionic polymer is a conductive polymer. When it is used to make a conductive film covering a conductive substrate to form a conductive structure, the zwitterionic polymer has high stability and is not easy to peel off. Also, since the monomers used to synthesize the polymer have two zwitterionic groups, compared with a conductive polymer synthesized from monomers having a single zwitterionic group, the present disclosure requires only a small amount of monomers to synthesize a zwitterionic polymer with sufficient anti-biofouling ability, thereby greatly reducing production costs and improving the hydrophilicity of the polymer. The conductive film of the present disclosure can be applied in various devices that require anti-biofouling (such as implantable medical devices) to cover the surface of its electrodes or probes, so as to prolong its service life. Moreover, the conductive film may not cause a significant increase in the impedance of the electrode and can avoid affecting its detection signal.

The present disclosure provides a zwitterionic compound having a structure of following formula (1), formula (2), formula (3), or formula (4):

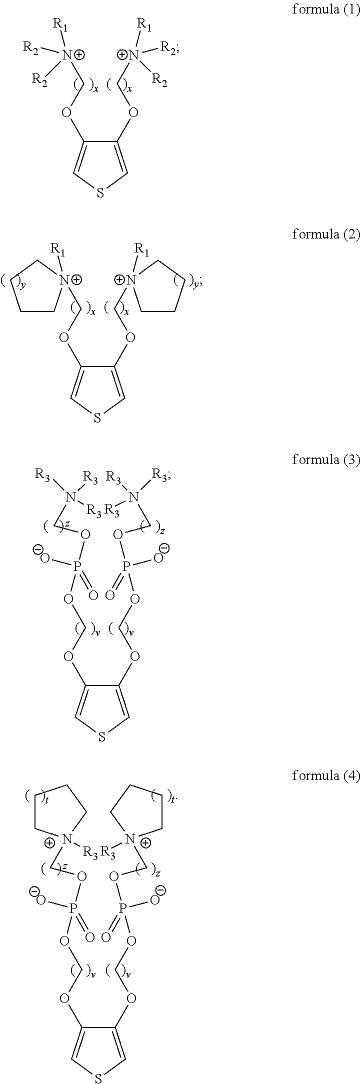

R₁ is —O⁻, —(CH₂)ₐSO₃⁻, or —(CH₂)_{b}CO₂⁻, a is 1 to 10, and b is 1 to 10, a and b are, for example, 1, 2, 3, 4, 5, 6, 7, 8, 9, or 10. R₂ and R₃ are each independently an alkyl group or a phenyl group. The alkyl group is, for example, a straight chain alkyl group or a branched chain alkyl group, and has a carbon number of, for example, 1-20, such as 1, 2, 3, 4, 5, 6, 7, 8, 9, 10, 11, 12, 13, 14, 15, 16, 17, 18, 19, or 20. x, z, and v are each independently 1 to 20, such as 1, 2, 3, 4, 5, 6, 7, 8, 9, 10, 11, 12, 13, 14, 15, 16, 17, 18, 19, or 20. y and t are each independently 0 to 10, such as 0, 1, 2, 3, 4, 5, 6, 7, 8, 9, or 10. The zwitterionic compound of the present disclosure has a thiophene ring with substituent positions 3 and 4 respectively having a zwitterionic group. As shown in formulas (1) and (2), the zwitterionic groups include trialkylamine N-oxide type, sulfobetaine (SB) type, or carboxybetaine (CB) type group. As shown in formulas (3) and (4), the zwitterionic groups include phosphobetaine (PB) type group.

In some embodiments, the zwitterionic compound has the structure of formula (1), R₁ is —O⁻, R₂ is a methyl group, and x is 2. Specifically, the zwitterionic compound has a structure of formula (1A) below. In some embodiments, the zwitterionic compound has the structure of formula (1), R₁ is —(CH₂)ₐSO₃⁻, a is 3, R₂ is a methyl group, and x is 2. Specifically, the zwitterionic compound has a structure of formula (11B) below. Comparing the following structures, it can be seen that the structure of formula (1A) contains short-chain functional groups, and the structure of formula (1B) contains long-chain functional groups as follows:

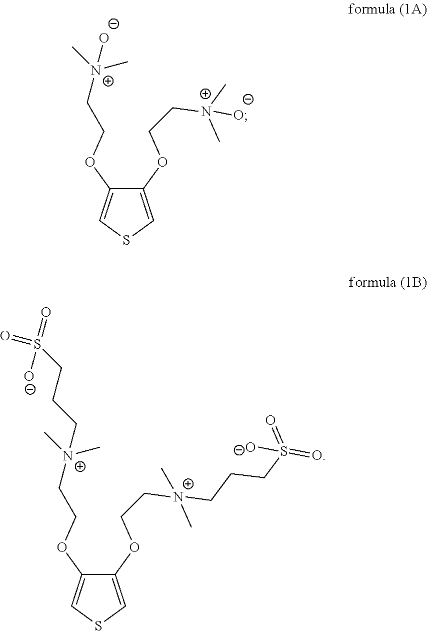

The present disclosure provides a method of producing a zwitterionic compound. The method includes reacting an oxidizing agent, a lactone, or a sultone with a reactant to obtain the zwitterionic compound having the structure of formula (1) or formula (2), in which the reactant has a structure of following formula (9) or formula (10):

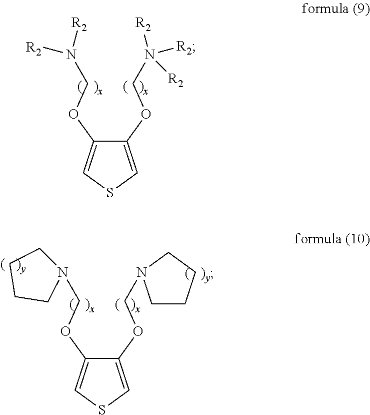

In some embodiments, the oxidizing agent includes meta-chloroperoxybenzoic acid (mCPBA), hydrogen peroxide, or a combination thereof. After the reaction, R₁ of the zwitterionic compound having the structure of formula (1) or formula (2) is —O⁻. In some embodiments, reacting the oxidizing agent with the reactant is performed at a temperature of 20° C. to 35° C., such as 20, 22, 24, 26, 28, 30, 32, 34, or 35° C.

In some embodiments, the lactone is reacted with the reactant, and the lactone has a carbon number of 2 to 11. After the lactone is ring-opened, it is connected to the nitrogen (N) atom of the reactant to form a group —(CH₂)_{b}CO₂⁻, where b is 1 to 10. In some embodiments, the sultone is reacted with the reactant, and the sultone has a carbon number of 1 to 10. After the sultone is ring-opened, it is connected to the nitrogen (N) atom of the reactant to form a group —(CH₂)ₐSO₃⁻, where a is 1 to 10. In some embodiments, reacting the lactone or the sultone with the reactant is performed at a temperature of 40° C. to 60° C., such as 40, 45, 50, 55, or 60° C.

The present disclosure provides another method of producing a zwitterionic compound to obtain the zwitterionic compound having the structure of formula (3) or formula (4). The method includes the following operations. In operation (1), 3,4-dibromothiophene is reacted with a diol

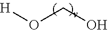

to form a first compound. During the reaction, the 3,4-dibromothiophene and the diol can be dissolved in a solvent (such as pyridine). In some embodiments, the operation (1) is carried out in an alkaline environment. For example, potassium tert-butoxide (t-BuOK) can be added to the solvent. In some embodiments, a catalyst, copper(I) iodide (CuI), can be added to the solvent. In some embodiments, the reaction formula is as follows:

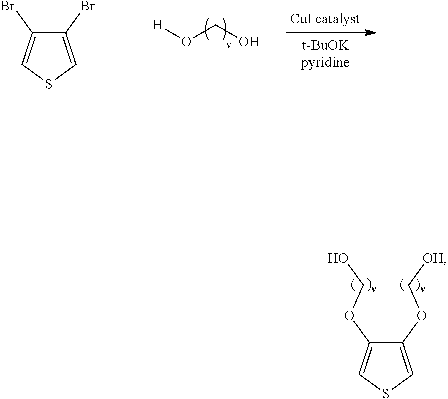

and the product is the first compound.
In operation (2), the first compound is reacted with

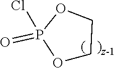

to form a second compound. During the reaction, the first compound and

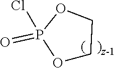

can be dissolved in a solvent (such as acetonitrile (CH₃CN)). In some embodiments, the operation (2) is carried out in an alkaline environment. For example, triethylamine can be added to the solvent. In some embodiments, the reaction formula is as follows:

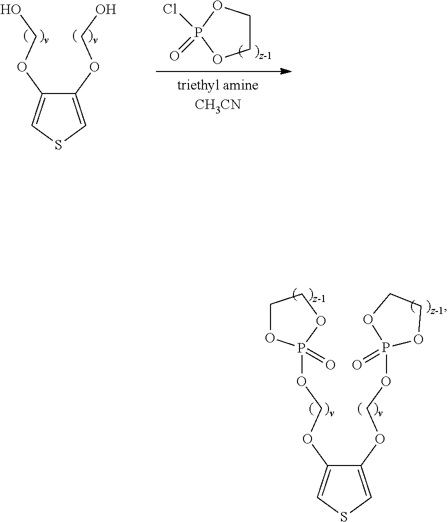

and the product is the second compound.
In operation (3), the second compound is reacted with an amine to obtain a third compound, in which the amine has a structure of

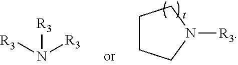

During the reaction, the second compound and the amine can be dissolved in a solvent (such as acetonitrile (CH₃CN)). If the structure of the amine is

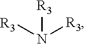

the third compound is the zwitterionic compound having the structure of formula (3). If the structure of the amine is

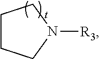

the third compound is the zwitterionic compound having the structure of formula (4). In some embodiments, the reaction formula is as follows:

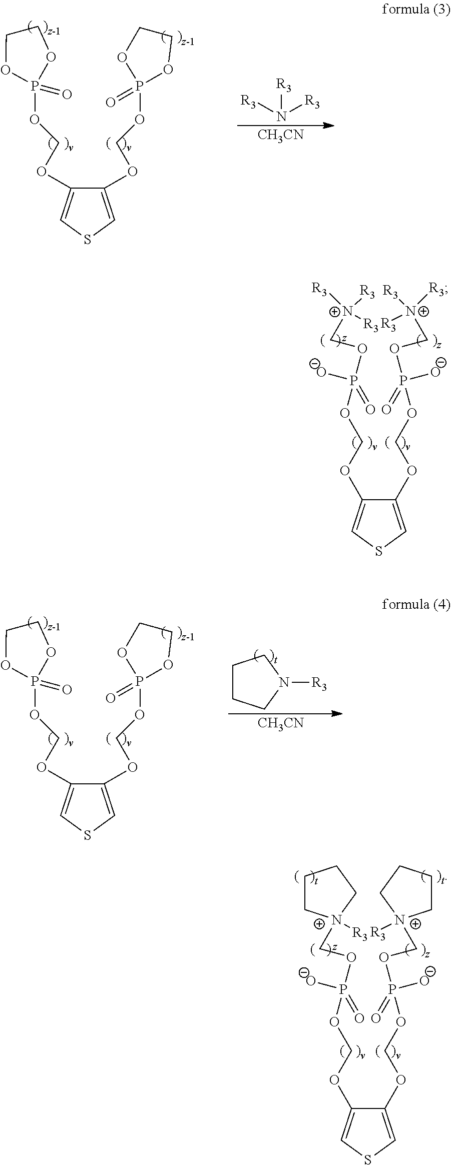

As can be seen from the above, the present disclosure provides the methods for producing the zwitterionic compounds with a simple preparation process and simple reaction conditions. Since the materials (zwitterionic derivatives) used for synthesis has low price, the zwitterionic compounds of the present disclosure have the advantage of low cost. For example, they are cheaper than 3,4-ethylenedioxythiophene (EDOT). Therefore, the zwitterionic compounds of the present disclosure and their manufacturing methods have great potential in industrial application.

The present disclosure provides a zwitterionic polymer including a chain segment formed by the zwitterionic compound having the formula (1), the zwitterionic compound having the formula (2), the zwitterionic compound having the formula (3), the zwitterionic compound having the formula (4), of any of the above embodiments, or combinations thereof. The zwitterionic polymer has a structure of following formula (5), formula (6), formula (7), formula (8), or combinations thereof:

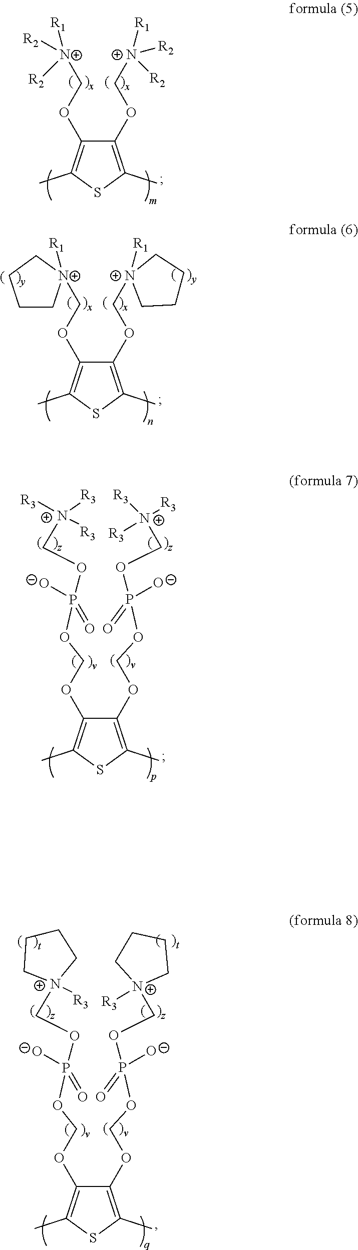

in which m is 1 to 10000, n is 1 to 10000, p is 1 to 10000, and q is 1 to 10000. m, n, p, and q can be independently 1, 100, 1000, 2000, 3000, 4000, 5000, 6000, 7000, 8000, 9000, or 10000. When m, n, p, and q are 1, the above-mentioned formula (5), formula (6), formula (7), and formula (8) are respectively a monomer unit in the zwitterionic polymer. In some embodiments, the zwitterionic polymer is formed by a composition including the zwitterionic compound having the structure of formula (1), the zwitterionic compound having the structure of formula (2), the zwitterionic compound having the structure of formula (3), the zwitterionic compound having the structure of formula (4), or combinations thereof. In some embodiments, the zwitterionic polymer is a copolymer, for example, a random copolymer or a block copolymer. In some embodiments, the zwitterionic polymer is polymerized from a composition including the zwitterionic compounds having formula (1) and formula (2). The zwitterionic polymer includes a chain segment having formula (5) and formula (6), so it is a copolymer. In some embodiments, the zwitterionic polymer is polymerized from a composition including the zwitterionic compounds having formula (3) and formula (4). The zwitterionic polymer includes a chain segment having the structures of formula (7) and formula (8), so it is a copolymer. In some embodiments, the zwitterionic polymer of the present disclosure is formed by polymerizing the zwitterionic compounds of any of the foregoing embodiments. For example, the zwitterionic polymer consists essentially of the structures of formula (5), consists essentially of the structures of formula (6), consists essentially of the structures of formula (7), or consists essentially of the structures of formula (8).

As can be seen from the above, the monomers having formula (1), formula (2), formula (3), or formula (4) can be polymerized to obtain the zwitterionic polymer having formula (5), formula (6), formula (7), or formula (8), respectively.

In some embodiments, the aforementioned zwitterionic compounds having formula (1A) or formula (1B) can be used as monomers for synthesizing a polymer. A polymer having a structure of formula (5A) or formula (5B) as follows can be synthesized:

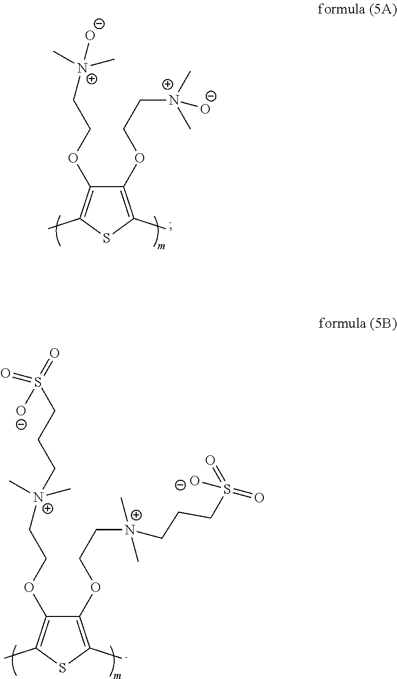

In some embodiments, the polymerization reaction includes electrochemical polymerization, thermal polymerization, photopolymerization, free radical polymerization, chemical catalysis polymerization, or enzyme catalysis polymerization. For example, the electrochemical polymerization includes the following operations. The monomers having formula (1), the monomers having formula (2), the monomers having formula (3), the monomers having formula (4), or combinations thereof are dissolved in an electrolyte to form a monomer solution. The solvent of the electrolyte includes, for example, acetonitrile, and the solute includes, for example, lithium perchlorate (LiClO₄) and sodium trimethylsilyl propyl sulfonate (DSS). Next, the polymerization of the monomers in the monomer solution is induced by cyclic voltammetry, in which a conductive substrate is used as a working electrode, and the reference electrode is, for example, Ag/Ag⁺. The voltage is, for example, 0.2 V to 1.2 V. The monomers are polymerized to form a conductive film (also called a polymer film) to cover the conductive substrate.

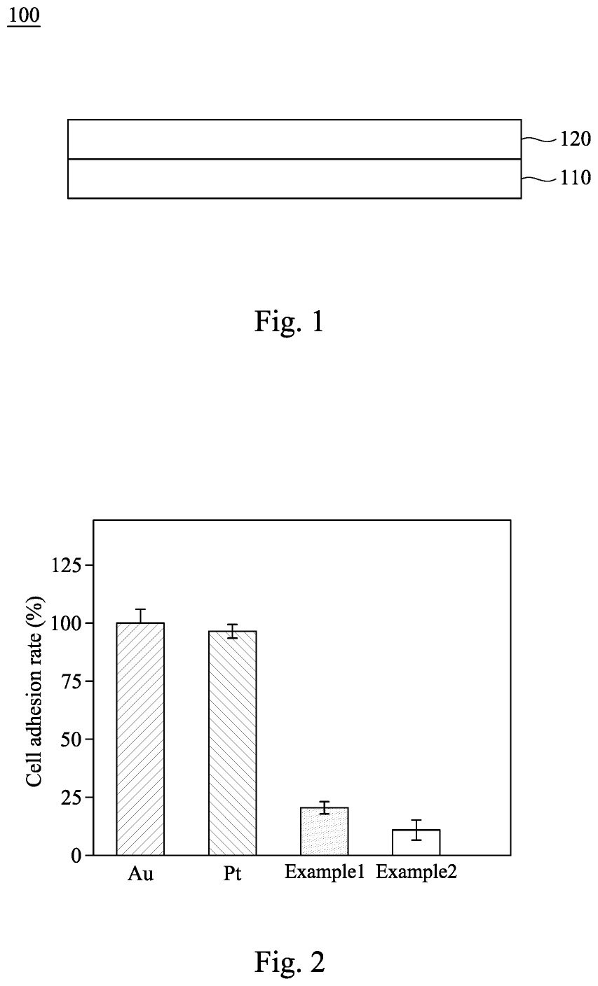

Please refer to FIG. 1. The present disclosure provides a conductive structure 100 including a conductive substrate 110 and a conductive film 120. The conductive film 120 covers the conductive substrate 110. In other words, the surface of the conductive substrate 110 is modified by the conductive film 120. The conductive substrate 110 includes a metal, an alloy, a metal oxide, a semiconductor material, a carbon material, or combinations thereof. The conductive substrate 110 is, for example, an electrode or a probe of an implantable medical device. In some embodiments, the material of the conductive substrate 110 includes gold (Au), platinum (Pt), aluminum (Al), iridium (Ir), titanium (Ti), steel, stainless steel, gold alloy, platinum alloy, platinum alloy, aluminum alloy, iridium alloy, titanium alloy, or combinations thereof, but not limited thereto. The gold and platinum have the characteristics of high conductivity, ductility, biocompatibility, and corrosion resistance, so they are considered as preferred materials for making probes or electrodes for in vivo detection. In some embodiments, the material of the conductive substrate 110 includes a conductive oxide, a carbon material, or a combination thereof. For example, the conductive oxide may include indium tin oxide (ITO), fluorine-doped tin oxide (FTO), tin oxide (SnO), indium zinc oxide (IZO), indium gallium zinc oxide (IGZO), indium tin zinc oxide (ITZO), other suitable conductive oxides, or combinations thereof, but not limited thereto. The carbon material may include conductive graphite, carbon black, carbon nanotubes, graphene, or combinations thereof. In some embodiments, the semiconductor material includes silicon (Si).

The conductive film 120 includes the zwitterionic polymer of any of the aforementioned embodiments, and has good conductivity, stability, and anti-biofouling ability. In some embodiments, the zwitterionic compounds of any of the aforementioned embodiments are polymerized on the surface of the conductive substrate 110, and thus the formed zwitterionic polymers are directly attached to the surface of the conductive substrate 110, thereby forming the conductive film 120. For example, the polymerization reaction is electrochemical polymerization.

The following describes the features of the present disclosure more specifically with reference to Examples 1 to 4. Although the following embodiments are described, the materials, their amounts and ratios, processing details, processing procedures, etc., may be appropriately varied without exceeding the scope of the present disclosure. Accordingly, this disclosure should not be interpreted restrictively by the embodiments described below.

### Example 1: Synthesis of the Zwitterionic Compound Having the Structure of Formula (1A)

First, please refer to the following reaction process 1.

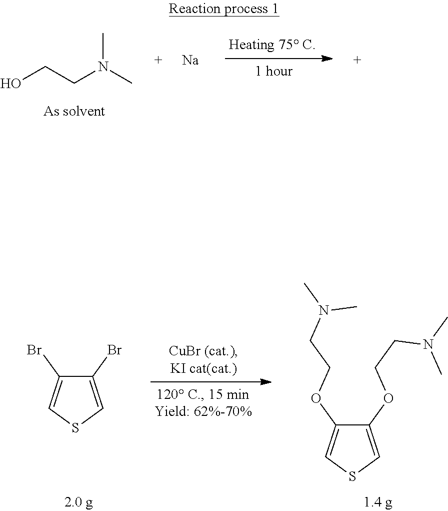

In step 1, 2-(dimethylamino)ethanol (10 mL) was used as a solvent in a 50 mL double-necked round-bottom flask equipped with a condenser and heated to 75° C. under nitrogen. In step 2, sodium metal (0.76 g, 33.3 mmol) was added within 1 hour until it was completely dissolved at 70° C. In step 3, 3,4-dibromothiophene (2.0 g, 8.33 mmol) was added at 70° C. and heated to 120° C. In step 4, 0.2 equivalents of copper(I) bromide (CuBr) and potassium iodide (KI) were used as catalysts at 120° C., and the reaction time is 15 minutes. In step 5, after stirring for 5 minutes, effervescence was observed. Thin-layer chromatography (TLC) was performed to visualize that the starting material had disappeared, and a new polarity point was observed. In step 6, the reaction was cooled to room temperature and purified by column chromatography using dichloromethane (DCM) having 10 wt %-15 wt % of methanol (MeOH) and 2 wt % of an ammonia solution. The yield was 62% to 70%.

The above product produced in the reaction process 1 of Example 1 was used as a reactant to synthesize the zwitterionic compound having the structure of formula (1A). Please refer to the following reaction process 2.

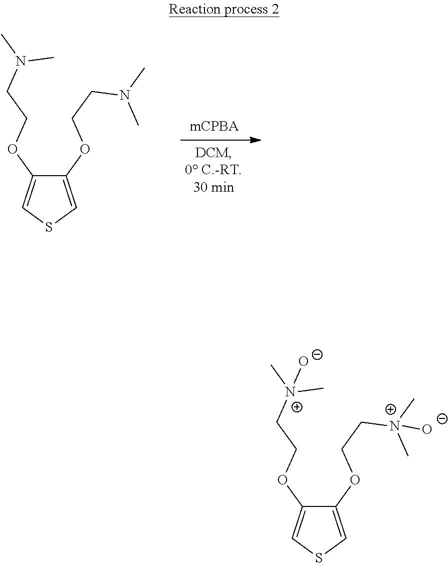

The product (0.2 g) produced in the reaction process 1, dichloromethane (DCM), and meta-chloroperoxybenzoic acid (mCPBA) were mixed at the reaction temperature of 0° C. to room temperature (RT) (RT is, for example, 20° C. to 30° C.) to obtain a yellow glassy solid product (120 mg, yield of about 61%), i.e., the zwitterionic compound having the structure of formula (1A), in which the reaction time is about 30 minutes. Alternatively, acetonitrile (ACN), methanol, or tert-butanol can be used instead of DCM as a solvent. In other words, the product produced from the reaction process 1 can also be dissolved in the ACN, methanol, or tert-butanol.

The data of nuclear magnetic resonance (NMR) spectra of the zwitterionic compound having the structure of formula (1A) are as follows: “the chemical shifts (δ) (unit: ppm) of ¹H-NMR (D₂O, 300 MHz) were 6.61 (1H, s), 6.45 (2H, s), 4.55 (4H, in), 3.76 (4H, m), 3.22 (12H, s).”

### Example 2: Synthesis of the Zwitterionic Compound Having the Structure of Formula (1B)

The above product produced in the reaction process 1 of Example 1 was used as a reactant to synthesize the zwitterionic compound having the structure of formula (1B). Please refer to the following reaction process 3.

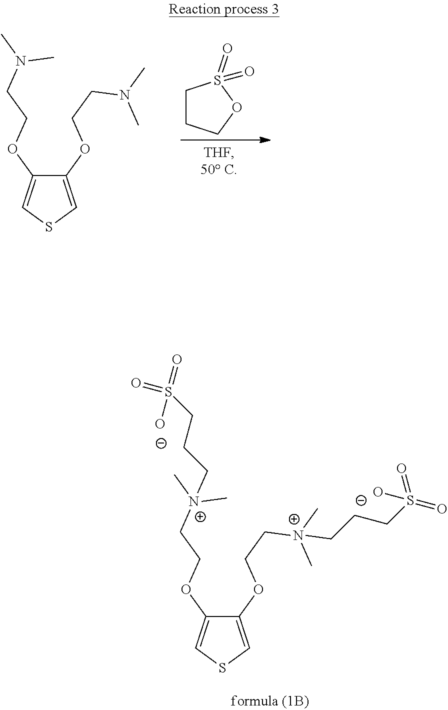

In step 1, the product (100 mg, 0.387 mmol) produced in the reaction process 1 was placed in dry tetrahydrofuran (THF). Acetonitrile (ACN) can also be used as a solvent instead of THF. In other words, the product produced from the reaction process 1 can also be dissolved in ACN. In step 2, 1,3-propane sultone (105 mg, 0.852 mmol) was added under nitrogen to form a reaction solution. In step 3, the reaction solution was heated to 50-55° C. (e.g., 50° C.) for 24 hours, and a solid precipitate was observed. The reaction time can be adjusted from 2 hours to 48 hours, e.g., 2, 10, 20, 30, 40, or 48 hours. In step 4, the precipitated solids were filtered and washed with a cold THF solution to obtain a beige solid product (120 mg, yield of about 60%), i.e., the zwitterionic compound having the structure of formula (1B).

The data of NMR spectra of the zwitterionic compound having the structure of formula (1B) are as follows: “the chemical shifts (δ) (unit: ppm) of ¹H-NMR (D₂O, 300 MHz) were 6.66 (2H, s), 4.55 (4H, m), 3.78 (4H, m), 3.49 (4H, m), 3.22 (12H, s), 2.96 (4H, m), 2.25 (4H, m).”

### Example 3: Hydrophilicity Measurement

The zwitterionic compounds of Example 1 were used as monomers for synthesizing polymers. A conductive film (containing polymers having the structure of formula (5A)) was formed on the surface of platinum by electrochemical polymerization, thereby forming modified platinum. The electrochemical polymerization includes the following operations. 0.1M lithium perchlorate (LiClO₄) and 0.05M trirmethylsilylpropyl sodium sulfonate (DSS) were dissolved in acetonitrile to prepare an electrolyte. The zwitterionic compounds having the formula (1A) were dissolved in the LiClO₄/DSS electrolyte to prepare a monomer solution (20 mM), and the dissolution was promoted by ultrasonic vibration for 5 to 10 minutes. Next, a cleaned unmodified platinum electrode was used as the working electrode in the monomer solution. The polymerization was induced by cyclic voltammetry (the voltage is 0.2 V to 1.2 V, the reference electrode is Ag/Ag⁺, the scan rate is 0.1 V/second), so that a polymer film was deposited on the surface of the platinum electrode. Afterwards, the platinum electrode with the polymer film was rinsed at least 3 times with acetonitrile and stored at 4° C. The zwitterionic compounds of Example 2 were used as monomers for synthesizing polymers. A conductive film (containing polymers having the structure of formula (5B)) was formed on the surface of gold by electrochemical polymerization, thereby forming modified gold. The electrochemical polymerization includes the following operations. 0.1M lithium perchlorate (LiClO₄) and 0.05M trimethylsilyipropyl sodium sulfonate (DSS) were dissolved in acetonitrile to prepare an electrolyte. The zwitterionic compounds having the formula (1B) were dissolved in the LiClO₄/DSS electrolyte to prepare a monomer solution (20 mM), and the dissolution was promoted by ultrasonic vibration for 5 to 10 minutes. Next, a cleaned unmodified gold electrode was used as the working electrode in the monomer solution. The polymerization was induced by cyclic voltammetry (the voltage is 0.2 V to 1.2 V, the reference electrode is Ag/Ag⁺, the scan rate is 0.1 V/seconds), so that a polymer film was deposited on the surface of the gold electrode. Afterwards, the gold electrode with the polymer film was rinsed at least 3 times with acetonitrile and stored at 4° C. In addition, the water droplet contact angles of the unmodified platinum and unmodified gold were measured. Please refer to Table 1 below for the measurement results. The smaller water droplet contact angle represents higher hydrophilicity of the surface. It can be seen from Table 1 that after the surfaces of platinum and gold were modified with the conductive films, their hydrophilicity are indeed significantly improved. The modified gold and platinum have good hydrophilicity.

TABLE 1

| | Unmodified | Modified | Unmodified | Modified |
|---|---|---|---|---|
| | platinum | platinum | gold | gold |
| Water droplet | 63.7 | 38.9 | 73.6 | 22.1 |
| contact angle | | | | |
| (degree) | | | | |

### Example 4: Measurement of Anti-Cell Adhesion Effect

The fouling conditions of the unmodified gold, the unmodified platinum, the modified platinum (modified with the compound of Example 1), and the modified gold (modified with the compound of Example 2) to L929 cells in the above Example 3 were observed. The L929 cells were incubated in a MEM incubation medium containing 10% fetal bovine serum and subjected to an attachment test. In a 24-well plate, 5×10⁵ cells/well of the L929 cells were incubated with the aforementioned metallic test pieces at 37° C. and in 5% CO₂ for 3 hours. Next, the cells were washed with phosphate buffered saline (PBS) to remove the unattached cells, and the test pieces were transferred to another clean well containing 500 ul of a cell incubation solution. The number of the attached cells was measured using PrestoBlue™ cell survival test solution (Invitrogen, A13262). After incubation at 37° C. and 5% CO₂ for 20 hours, 100 ul of the reactant supernatant was taken into a 96-well plate, and the optical density (O.D.) of the reaction solution was read using an enzyme immunoassay reader (using excitation/emission wavelengths of 535 nm/615 nm). The results are shown in FIG. 2. The absorbance of a wavelength of 570 nm was used to calculate the cell adhesion rate, and the cell adhesion rate is calculated as [(absorbance of sample group OD₅₇₀−absorbance of blank group OD₅₇₀)/(absorbance of control group OD₅₇₀−absorbance of blank group OD₅₇₀)]*100%. From the experimental results in FIG. 2, it can be seen that the adhesion of the gold electrode modified by the polymer film to the L929 cells is quite low (about ⅕- 1/10 of that of the unmodified gold electrode) compared with that of the unmodified metal electrode, and the adhesion of the platinum electrode modified by the polymer film to the L929 cells is also quite low. It can be seen that the platinum and gold electrodes modified with the conductive films are less likely to adhere to the L929 cells, which means that they have good resistance to cell adhesion.

In summary, the present disclosure provides a zwitterionic compound, a producing method thereof, a zwitterionic polymer thereof, and a conductive structure thereof. The reactants for synthesizing the zwitterionic compound are cheap, so the zwitterionic compound, the zwitterionic polymer, and the conductive structure have the advantage of low cost. Moreover, since the zwitterionic compound has two hydrophilic zwitterionic groups, the zwitterionic compound can have high hydrophilicity and high resistance to nonspecific biofouling. Since the zwitterionic compound contains a thiophene ring, the zwitterionic polymer has good electrical conductivity. Only a small amount of monomer is needed to synthesize the zwitterionic polymer with sufficient anti-biofouling ability, so the production cost can be greatly reduced, and the hydrophilicity of the polymer can be improved. The zwitterionic polymer of the disclosure can have high hydrophilicity, high conductivity, high anti-biofouling ability, and high biocompatibility, have low immunogenicity, and can be used in various devices that require anti-biofouling (such as implantable medical devices) to modify the surfaces of electrodes or probes of the devices. The zwitterionic polymer has high stability and is not easy to peel off, which can prolong the service life of the devices. According to the above, the present disclosure provides hydrophilic conductive materials with a simple preparation process, ultra-high hydrophilicity, low price, and high stability.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A zwitterionic compound, having a structure of following formula (1), formula (2), formula (3), or formula (4): 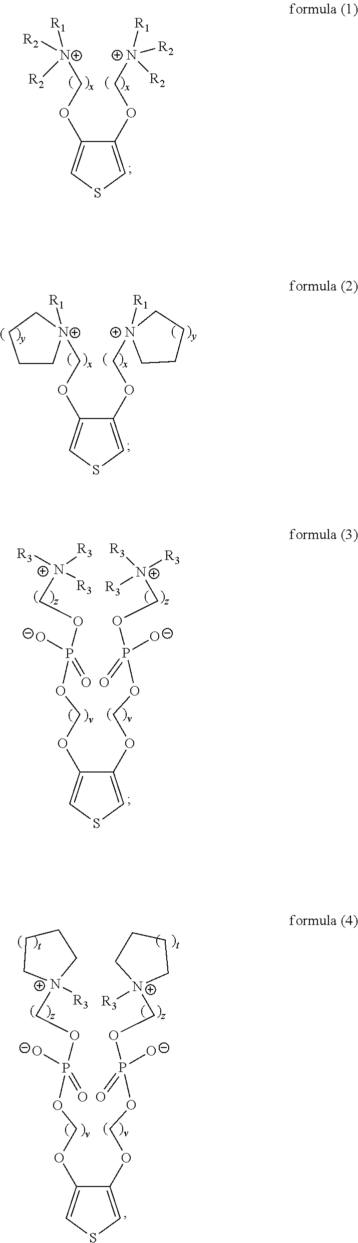
wherein R₁ is —O⁻, —(CH₂)ₐSO₃⁻, or —(CH₂)_{b}CO₂⁻, a is 1 to 10, b is 1 to 10, R₂ and R₃ are each independently an alkyl group or a phenyl group, x, z, and v are each independently 1 to 20, and y and t are each independently 0 to 10.

2. The zwitterionic compound as claimed in claim 1, wherein the zwitterionic compound has the structure of formula (1), R₁ is —O⁻, R₂ is a methyl group, and x is 2.

3. The zwitterionic compound as claimed in claim 1, wherein the zwitterionic compound has the structure of formula (1), R₁ is —(CH₂)ₐSO₃⁻, a is 3, R₂ is a methyl group, and x is 2.

4. A zwitterionic polymer, comprising a chain segment formed by the zwitterionic compound having the formula (1), the zwitterionic compound having the formula (2), the zwitterionic compound having the formula (3), the zwitterionic compound having the formula (4), or combinations thereof as claimed in claim 1, the zwitterionic polymer having a structure of following formula (5), formula (6), formula (7), formula (8), or combinations thereof: 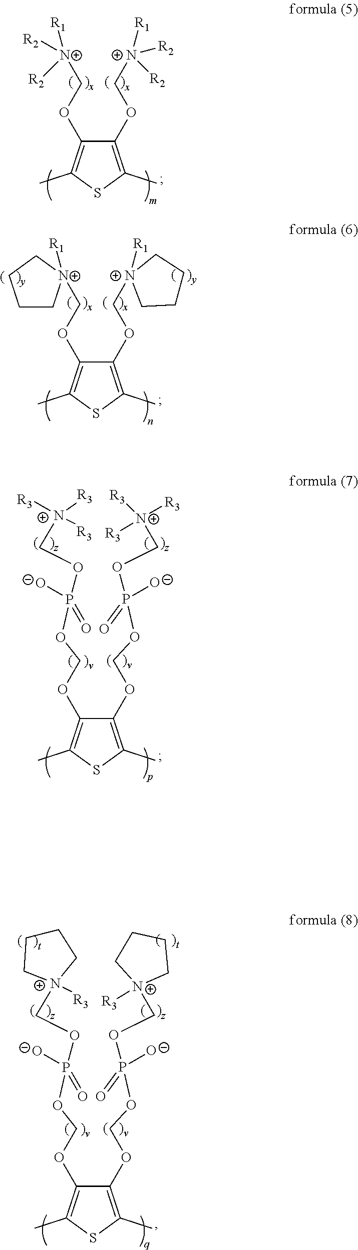
wherein m is 1 to 10000, n is 1 to 10000, p is 1 to 10000, and q is 1 to 10000.

5. The zwitterionic polymer as claimed in claim 4, wherein R₁ is —O⁻, R₂ is a methyl group, and x is 2.

6. The zwitterionic polymer as claimed in claim 4, wherein R₁ is —(CH₂)ₐSO₃⁻, a is 3, R₂ is a methyl group, and x is 2.

7. A conductive structure, comprising:
a conductive substrate comprising a metal, an alloy, a metal oxide, a semiconductor material, a carbon material, or combinations thereof; and
a conductive film covering the conductive substrate, wherein the conductive film comprises the zwitterionic polymer as claimed in claim 4.

8. A method of producing a zwitterionic compound, comprising:
reacting an oxidizing agent, a lactone, or a sultone with a reactant to obtain the zwitterionic compound having the structure of formula (1) or formula (2) as claimed in claim 1, wherein the reactant has a structure of following formula (9) or formula (10):

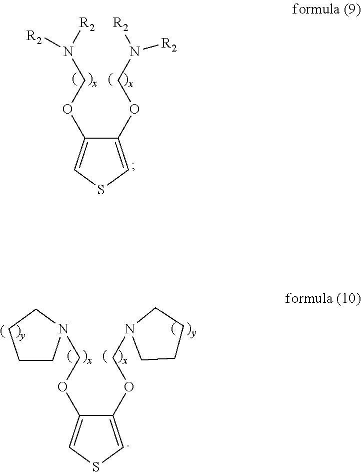

9. The method as claimed in claim 8, wherein the oxidizing agent comprises meta-chloroperoxybenzoic acid, hydrogen peroxide, or a combination thereof.

10. The method as claimed in claim 9, wherein reacting the oxidizing agent with the reactant is performed at a temperature of 20° C. to 35° C.

11. The method as claimed in claim 8, wherein when reacting the lactone or the sultone with the reactant, the lactone has a carbon number of 2 to 11, and the sultone has a carbon number of 1 to 10.

12. The method as claimed in claim 11, wherein reacting the lactone or the sultone with the reactant is performed at a temperature of 40° C. to 60° C.

13. The method as claimed in claim 8, wherein R₂ is a methyl group, and x is 2.

14. A method of producing a zwitterionic compound, comprising:
reacting 3,4-dibromothiophene with 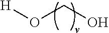
to form a first compound;
reacting the first compound with 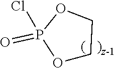
to form a second compound; and
reacting the second compound with an amine to obtain the zwitterionic compound having the structure of formula (3) or formula (4) as claimed in claim 1, wherein the amine has a structure of

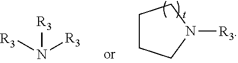

15. The method as claimed in claim 14, wherein reacting the 3,4-dibromothiophene with 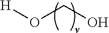
comprises:
dissolving the 3,4-dibromothiophene, 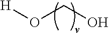
and a catalyst in a solvent, wherein the catalyst is copper(I) iodide.
